(19) United States
(12) Patent Application Publication
(10) Pub. No.: US 2026/0078410 A1
(43) Pub. Date: Mar. 19, 2026

(54) NANOSCOPIC PROJECTILES

(71) Applicants: Paul Westmeyer, Lancaster, PA (US); Renee Mazaheri, Lancaster, PA (US)

(72) Inventors: Paul Westmeyer, Lancaster, PA (US); Renee Mazaheri, Lancaster, PA (US)

(21) Appl. No.: 19/109,231

(22) PCT Filed: Sep. 7, 2023
(86) PCT No.: PCT/US2023/032140

§ 371 Date: Mar. 6, 2025

Related U.S. Application Data
(60) Provisional application No. 63/404,596, filed on Sep. 8, 2022.

(51) Int. Cl. C12N 15/89; C12N 15/01

(52) U.S. Cl. CPC C12N 15/895; C12N 15/01

(57) ABSTRACT

A system comprised of customized nanoscopic projectiles directed to target cells. Projectiles are metered into a central force accelerator, comprised of hundreds to thousands of concurrent channels. Asperities of the channels' surface, transferring momentum from the central force accelerator to the projectiles, are atomic level asperities. Projectiles penetrate to target cells, deliver chemicals, without using the organism's circulatory system, resulting in cells being altered; killed if cancerous, modified by genetic materials contained in projectiles, and enhanced in performance by projectiles loaded with content to assist in cellular purposes.

### CROSS-REFERENCE TO RELATED APPLICATIONS

The present invention is based upon and claims the benefit of provisional patent application No. 63/404,596, filed on Sep. 8, 2022.

### TECHNICAL FIELD

In one embodiment, the invention is a method of direct insertion of nanoparticles without using the circulatory system presents opportunities for novel cellular biochemical operations. Operational utility of direct insertion of nanoparticles can include drug development testing, and ultimately mass production, cancer treatment with existing compounds, and new compounds not viable for circulatory based treatment, or various genetic modifications of cellular functions.

### BACKGROUND OF THE INVENTION

Advances in biological sourced generate products are realistic as plants and microbes are modified. Individual cells are targets and these cells can be evaluated as biological factories. Temporary and potentially permanent corrections to genetic defects can be deployed into effected cells. Materials and drug developments can also benefit from injections of specialized projectiles, carrying genetic segments and other chemicals, into plants, microbes, and hybrid organisms. Projectiles can host chemical compounds, or segments of genetic materials for deployment to the nucleus or cytoplasm. Compounds can be new treatments or existing treatments. Expanding the amino acids is an obvious tool once genetic segments are deployed, thus interesting proteins can be fabricated in cells hosting the modified genetic materials.

Human genetic segments, and old and new compounds can be tools against cancer, disrupting the cancer cell reproduction cycle.

### BRIEF SUMMARY OF THE INVENTION

Cellular Penetrations are comprised of customized nanoscopic projectiles directed to target cells. Projectiles are metered into a central force accelerator, comprised of hundreds to thousands of concurrent channels. Asperities of the channels' surface, transferring momentum from the central force accelerator to the projectiles, are atomic level asperities. Projectiles penetrate to target cells, deliver chemicals, without using the organism's circulatory system, resulting in cells being altered; killed if cancerous, modified by genetic materials contained in projectiles, and enhanced in performance by projectiles loaded with content to assist in cellular purposes. Cellular purposes can be altered by genetic changes, potentially creating new industrial capacity for complex plant, plant-animal, and microbe synthesis of matter not naturally found in the organism prior to genetic alteration. Cells between the accelerator's channels exit ports and the target cells will experience puncture damage as nanoscopic projectile transit enroute to the target cells.

Volumetric deposition is selectable; two axes perpendicular to the projectiles' velocities provide an area definition, while depth is controlled by projectiles' momentum. Projectiles with larger length over diameter parameters may open a pathway for subsequent projectiles with different forms and masses. Projectiles containing healing compounds can be used to address damage done when projectiles penetrate healthy cells located between the machine's exit portals and the target cells.

Several areas of research are significant to this application; (1) generation of compounds from plant and microbe operators altered by projectile insertions into the microbes or plants, (2) permanently altering genomes in species, including humans, by projectiles causing changes in a cell nucleus' Deoxyribonucleic Acid (DNA), (3) temporarily alter versions of Ribonucleic Acid (RNA) in a cell's cytoplasm to generate a change, and (4) targeted attacks on organelles in the cell's cytoplasm with projectiles.

Projectiles will be accelerated by a classic central force machine, several are proposed. Techniques for accelerating nanometer-class objects are underlying preconditions to achieving the outcomes at cellular scales of interactions. Patents defining central force acceleration of macroscopic objects have been issued to the inventors. Three U.S. issued patents, and their international versions, are incorporated by reference. These are U.S. Pat. Nos. 7,013,988, 8,820,303, and 10,343,258. Additional central force technical materials are referenced and/or used. There are technical challenges to accelerating nanometer class objects that make the previous patents unusable as issued.

### BRIEF DESCRIPTION OF DRAWINGS

FIG. 1 shows a Multi-Walled Carbon Nantotube (MWCNT).

FIG. 2 shows layers of Graphene.

FIG. 3 shows functionalization constructs for CNT.

FIG. 4 shows images of highly polished metal surfaces.

FIG. 5 shows a typical animal cell.

FIG. 6 shows Ferritin and Apoferritin nanoparticles.

### DETAILED DESCRIPTION OF THE INVENTION

Surface properties of the accelerating structures of any of the previous patents or concepts will not be sufficiently smooth to actually achieve any meaningful acceleration of a nanometer-class projectile. These older ideas used polished surfaces resulting in the nanometer objects being subjected to surface irregularities larger than the nanoparticle. Asperities of most polished metal surfaces, nominally in micron, will ‘appear’ as mountains when compared to the nanoparticle. Between peaks of the asperities will be valleys, nanoparticles will become trapped as they accumulate in the valleys between peaks. Several solutions can be deployed to reduce asperities, including using optical telescope quality surfaces with piezo-electric actuators or selecting a material with atomic structures that fail to establish asperities; nanotubes and two-dimensional structures like Graphene. Augmenting the previous patents with these changes will require new designs.

Similarly, the flow control of small volume of nanoparticles, as small as one-billionth of an animal cell, defined as a sphere of roughly 30 microns diameter or equivalent cubic object of similar dimensions, requires an insertion control unlike anything in the previous patents. Addressing relatively small numbers of molecules or compounds, or composites of molecules and compounds, has inherent challenges, this is exaggerated by the subsequent acceleration. Gate controllers made from nanostructures will be necessary to generate accurate masses (volume of a compound).

Cellular and sub-cellular drugs development research, and subsequent treatments, are undergoing transformational work, with emphasis on nanometer biochemical processes. Understanding of the complexities of interplay at those dimensions is difficult and relies upon statistical assessments. As the processes are qualified and quantified, with ever-increasing complex experimentation, the opportunities for improving human health increase. Barriers of experimentation are plentiful, as found in the types and scope of professional articles in journals. Some of the journals of interest are medical journals, others are nanoscience centric, yet others are natural science covering topics like insect stingers and how they are constructed. There is no shortage of relevant materials to review, as the development processes advance the State-of-Art (SOA).

There are thousands of patents and patent applications related to topics of drug production, drug interactions, coopting nature to mass produce drugs or components in drugs, including 20 human used amino acids and several hundred additional amino acids (sometimes referred to as unnatural amino acid or UAA) to produce novel proteins. UAA usage nominally involves genetic alteration of microbes or plant species.

Wet chemistry is a mass production process. Some newer strategies employ microbes and plants as ‘factory workers’ inside production facilities. Microbe and plant strategies rely upon some natural or quasi-natural processes being discovered and made into useful elements of an overall drug/compound production. Improving on the probability of having a viable microbe or plant ‘factory’ is an important aspect of the processes being developed in this application. Adding to the cellular activities without altering the genome, which creates a genetically modified organism (GMO), will require a continuous insertion of projectiles. Projectiles can host ‘instruction’ and/or processes' ingredients.

Altering genomes is a permanent effect. Nucleus changes are required, a step beyond the previous continuous projectile insertion, this is how GMO are created. GMO foods are already researched, but the efficiency of experimentation might become significantly faster as more nuclei can be altered. These permanent changes are passed along to the next generations.

While the permanent correction for humans might relieve impacts of disease there no suggestion of changing the genetics associate with the next generation, this application merely suggest the correction can be applied wherever it is required, no selection of genetic traits is conferred. Projectiles also advance the potential usage of genetic ‘correction’ for known genetic disease in humans.

With advances in engineering tools the State-of-Art (SOA) for nanometer medical research can be advanced rapidly. Nanoparticles are commonly used in engineering, and in biological sciences including medicine and pharmacy fields. Unoxidized nano-metal is typically sealed in an oxide layer; aluminum-oxide (Al₂O₃) encasing pure aluminum (Al) is an example. Breaking the encasement can expose the unoxidized aluminum to oxygen, resulting in an exothermic reaction.

Nanoparticles are used in medicine, with associate risk benefit analysis for each application of nanoparticles. Nanoparticle drugs are provided intravenously; circulatory system. Near surface applications are via needles or Needle Free Injection Technology (NFIT), drugs are absorbed and transferred via local circulation as opposed to whole body circulation system. The cross-section of the needle or NFIT jet are significantly larger than a cell's cross section and damage is significant to cells. Direct insertion into a cell or group of cells, without a circulatory action, is not currently in the options for medical treatments.

Access to a specific cell, or cell cluster, by a projectile is a momentum problem. A proton is a projectile used in proton-therapy. The proton's penetration is defined by the dynamics of momentum between the local mass and the proton. Penetration depth for a nanoscopic projectile is still a dynamics problem with the classic parameters; including the mass, velocity, cross-sectional interaction, and characterization of the medium being penetrated. Nanoparticles are much more massive than a single proton. At the other extreme, bullets enter bodies too. Nanoparticles are between the proton and a bullet, in terms of mass. Obviously, plant and microbes have fewer cells in thickness thus the depth is smaller than a human.

A secondary practical matter is the continuous nature of generating momentum for sufficient nanoparticles as medicines, or as precursor activity to fabricate medicines, or to generate test articles to create new medicines, etc. Going from a ‘proof-of-concept’ to a fully functional operational assembly line is a classic engineering function.

Medical research teams need engineering tools for accelerating large numbers, many millions to many billions, of nanoparticles per treatment session. Massively parallel acceleration channels are the engineering solution to achieving the large numbers; 3.6 billion groups of nanoparticles accelerated in one hour (3,600 seconds) requires one million parallel acceleration cycles; this can be further defined as parallel channels operating at sub-second cycles. The parallel channels are further defined as two-dimensional array technology. The trade space is finite, but the scaling is very much within the engineering SOA.

Coopting a microbe or plant or a larger animal host to create drugs is not a new idea. Numerous ideas are being explored to replace wet chemistry (large factory chemical production) with natural hosts that ‘do the work’ as part of their life cycle. Extreme ideas include expansion of the amino acids available for product generation. Gene spicing has been accomplished in specific organisms, even to cross-over the plant-animal boundary. New genetic ‘breeds’ are readily found in consumable food, the ‘Genetically Modified Organism’ (GMO). The potential value to mass production activities includes; enhancing the pace of experimental work by parallel testing billions of cells in short periods of time, and second, the same process can be applied to larger scales once a product line is defined.

Consider a researcher doing tedious work to fabricate test sequences, potentially days or weeks of precise wet chemistry, requiring access to billions of cells. With a direct insertion device potentially all of this tedious work can be sourced to a mechanical tool capable of accessing billions of test article cells in a few hours. Meanwhile the wet chemistry is sidestepped, thus reducing the complexity and error potential of the experiment (especially critical when retested by a non-advocate team validating the research products). As an example, projectiles hosting amino acid compounds, and genetic code projectiles can produce unique proteins by the cell's internal functions.

Medical specialists are hampered by the delivery of treatments to individual cells of interest, this is especially true of genetics. When a genetic disease is narrowed down to a specific coding flaw the research team is elated. Medicinal benefit occurs when the research is reduced to practice, as a treatment to the cells where the disease is expressed. Those intervening steps between gene isolation and a treatment are difficult to achieve, some of the difficulties include the means to get the treatment into the necessary body parts.

Genetic editing has advanced to be a ‘State-of-Art’. Techniques for adding or removing or altering a small segment of any genome are commonplace. Three techniques are: clustered regularly interspaced short palindromic repeats (CRISPR), transcription activator-like effector nucleases (TALENs) and zinc-finger nucleases (ZFNs).

Creating projectiles of Deoxyribonucleic Acid (DNA), Ribonucleic Acid (RNA), messenger RNA (mRNA), or transfer RNA (tRNA) is a subject of research. These projectiles are just a subset of complex chemistry under consideration. Delivery systems being developed by researcher using NFIT for shallow skin penetration or intravenous for whole organism deployment, both require unique chemistry to reach the target cells, and achieve sufficient medicine uptake to generate a meaningful treatment.

Significant progress has been made into defining genetic flaws and isolating repair codes, but not into their packaging and delivery to billions of cells. Packaging is a step in a multi-step process to achieve a desired treatment, once delivered the treatment needs to be integrated into the cellular processes. Thus, packaging is more than just the genetic code, each package also needs the tools to engage the cellular processes. Nucleus safeguards against disease also work to stop other additive materials from altering the contents of the nucleus. Non-nucleus cellular engagement is non-trivial, and a preamble to nucleus engagement.

Two examples of a known genetic are Cystic Fibrosis (CF) and Phenylketonuria (PKU). Both these candidate examples have known genetic signatures. CF impacts two bodily functions, the lungs and digestion, airways and oral ingestion are independent pathways beyond the circulatory system delivery; however. Persons living with CF have seen a great expansion in life expectancy, and improved quality of life. PKU has no such alternative delivery strategies and must rely upon circulatory system to provide genetic correction to billions of cells needing updated DNA or mRNA (a temporary fix). Neither of these diseases have a means to repair the genetic flaws. Many other genetic diseases with known or knowable genetic treatment will remain partially solved, the coding fix is known, and await packaging and a delivery system.

Chemotherapy is nominally dependent upon the circulatory system to reach the targeted cells, radiological treatments can be circulatory-based or implanting of radioactivate materials (radioactive wire placed near the cancer growth) or energetic particle bombardments. Unfortunately, the circulatory technique (chemo and radiological) impacts the whole human, limiting choices of chemistry. Radiological implants are more localized, but still impact healthy cells. Energetic particles, especially protons, are highly localized, with success for most people. Ideally some common packaging and a localized delivery system will be achieved at the cellular level to address these medical needs. Adding to these options is worthy of research.

One creative circulatory delivery system is a modified ‘host’ virus. A twist of irony wherein the classic host-virus relationship has been transformed, the virus has been genetically altered to be a host, yet acts like a virus. Still a circulatory system action and whole patient is engaged, not an ideal solution.

Cellular dimensions create challenges to packaging and delivery systems. Additionally, the number of cells needing treatment can be in the billions. Cancer is commonly referenced by stages and tumor size (pea is 1 centimeter, grape is 3 centimeters etc.) and the number of cells in the tumor can be estimated by assigning a volume to each cancer cell based upon rough dimensions of the cell type.

Existing mechanical systems such as needles and Needle Free Injection Technologies (NFIT) don't scale down to cellular dimensions, not alone address the quantities (billions of cells, and small numbers of projectiles).

Size matters, proton therapy (no packing required) works because the projectile is orders-of-magnitude smaller than the target cell. Damage to non-target is deemed acceptable, mostly because no alternative exists. Quantities of protons are not an issue, and volumetric deposition controls with Electro-Magnetics (EM) are straightforward. Other charged particles, electrons and alpha particles, can be used as well.

Scaling upward from the Electro-Magnetic (EM) acceleration of protons (in Proton Therapy) to larger molecular projectiles is problematic, and no commercialization is known. Molecules generally have no embedded EM component capability, barring classic molecular polarity (water being an example). Altering the molecular composition to host charged nodes at the nanometer scale, to take advantage of EM acceleration techniques, would be an interesting concept, if possible. A single proton carries a plus ‘1’ charge, and a very small mass.

Likewise, scaling downward from EM levitated rail, or military ‘railgun’ EM systems, is not practical. In both those larger object systems EM components hosting charge are embedded in the object being accelerated.

However, scaling downward in particle size from commercial mechanical central force driven acceleration of macroscopic particles is credible, but challenging. Several Space Launch (to place objects into earth orbit) mechanical designs have been significantly altered to smaller macroscopic projectiles, and with shorter ranges. Designs using simple spinning, simple gyrating, more complicated gyrations using multiple frequencies and gyration radii and rephasing histories, and even combined spin-gyrate mechanical acceleration have been developed for small macroscopic work; sandblasting is a classic example. These small macroscopic projectiles, used in commercial applications, are substantially more massive and dimensionally larger than a single cell. This application takes the grandness of small to the near molecular scale for a projectile.

Advancing the acceleration technique for macroscopic projectiles, to a State-of-the-Art for microscopic and nanoscopic projectiles, is the delivery system being coupled to the genetics research, and also chemotherapy, and potentially to fundamental materials development research. Projectiles need to be very small microscopic (sub-cellular) and preferably nanoscopic. Much like proton therapy the smaller the projectile the less unintended damages to healthy cells impacted by a fast-moving projectile transiting the healthy cells.

Nanometer class objects are well known, including biological compounds.

These inventors have extensive knowledge of macroscopic work with their previously issued central force patents for gyrating systems in multiple configurations, spinning, and spin-gyrate designs, and related central force patents issued to other inventors in this field. Previous designs for gyrate and spin-gyrate macroscopic projectiles will require modifications for surface properties of the accelerator's surfaces in contact with the projectiles, precise targeting of a multitude of targets within a defined volume, and quantities necessary for cellular utility. Dimensional qualities, absolute size and tolerancing of all the previous designs are incongruent with the logic of precision cellular targeting, none of the previous work taught microscopic or nanoscopic short-range targeting. Additionally, many macroscopic projectiles' gyrate designs do not address necessary pluralities of channels for directing media or projectiles in a simultaneous fashion with coordination of targets. Multiple channels gyrating and spinning, and spin-gyrate designs exist, just not at scales necessary to achieve billions of projectiles firing within a medical treatment timeline.

Two parameters favor nanoscopic systems; low overall mass, and low power consumption.

Likewise, the projectiles themselves have to undergo transformation from macroscopic (yet small) to what the genetic and chemotherapy research teams have created as medicinal treatments. Molecules are nanometer class. Treatment molecules can be either left ‘bare’ or encased.

Morphing both the accelerator and the projectiles are new ideas and has fostered a frontier of much more than just a simple cure for one or two diseases.

Oregon State University (OSU) opened a Genetic Code Expansion (GCE) Center in Feb. 2022, with emphasis on expanding the 20 amino acids to over 200 amino acids, allowing for more building blocks for proteins, proteins with novel consequences to organisms. OSU's Unnatural Protein Facility has been active for years.

One aspect of unnatural proteins work is the synthesis from building blocks of smaller amino acids. Tricking a microbe or plant into accepting new genetic directions is very dependent upon complex chemistry, much of which is only being discovered. If the complex chemistry can be augmented with a less complex configuration of ‘instruction set’ tools at scales with realistic utility (industrial techniques), then expediting the results of experimentation and ultimately drug production is likely to be enhanced. Mass injector strategy into billions of cells, not reliant upon complex chemistry to gain control over an organism's cellular processes, is the approach being proposed.

Bio-engineering a non-circulatory delivery system and compatible projectiles of advanced biochemistry in an open-ended science activity.

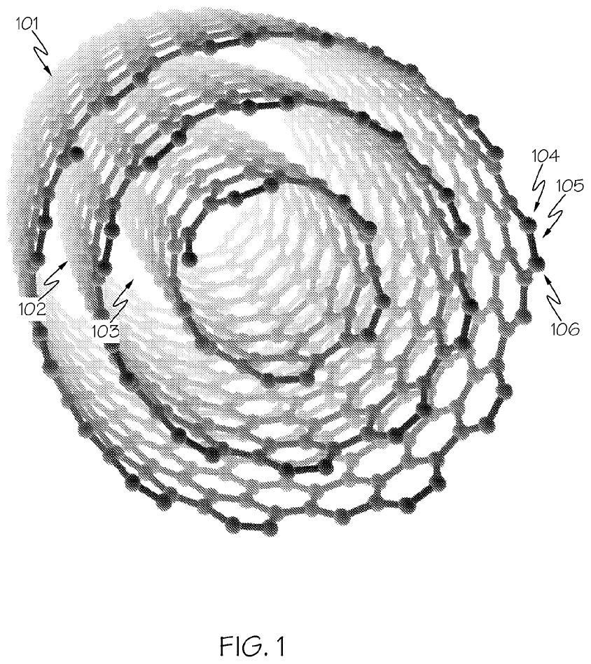

FIG. 1 shows a Multi-Walled Carbon Nanotube (MWCNT) structure; technically a molecule, not a compound, there is only one atom type in the entire structure; carbon. This is a classic modelling technique used in introductory chemistry; carbon atoms are nodes, and stick depiction of covalent bonding between carbon atoms. Actual sizes are greatly exaggerated. Techniques of this nature are foundational in teaching chemistry. Each carbon atom node is represented by spherical objects, objects 104 and 106 are two of the many carbon atoms, and the carbon atoms' electron covalent bonds are represented by the sticks, object 105 is the bond between carbon atoms defined as objects 104 and 106.

FIG. 1 has three tubular structures, object 101 as the largest diameter carbon nanotube (CNT), and object 102 as the intermediate diameter CNT, and object 103 as the smallest diameter CNT, as one structure Multi-Walled Carbon Nanotube (MWCNT). The innermost tube structure is smaller in diameter than the middle tube, and the largest diameter tube is bigger than the smaller tubes' diameters.

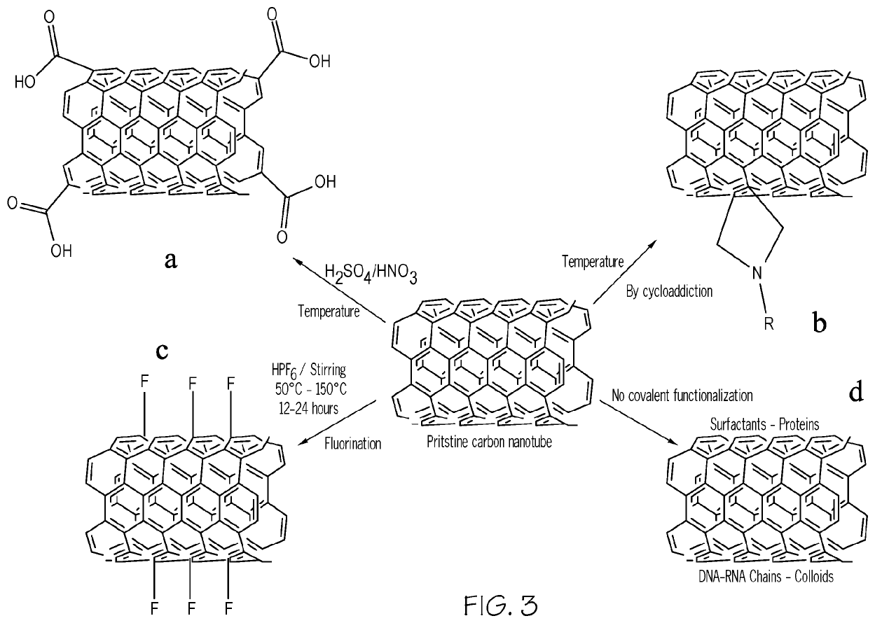

FIG. 3 shows five layers of Graphene, as individual nearly 2-dimensional molecular structures (not a compound), objects 201 through 205. Like the MWCNT of FIG. 1, this representation uses the spherical nodes, objects 206 and 208, for the carbon atoms, and a stick for the covalent bond, object 207, between nodes 206 and 208. Similar to the MWCNTs, the layers are isolated from each other, represented by a lack of electrons' bonding. There is a third dimension in the surface of Graphene, caused by the bonds' angles. Due to the atomic dimensionality the third dimension is commonly ignored, and Graphene is considered a two-dimensional array of carbon atoms. The height of the third dimension is practically lost when compared to the planar dimensions, which can become macroscopic in nature.

Geometrically speaking, Graphene becomes carbon nanotubes when the surface is rolled.

FIG. 3 shows a few examples of effectors with CNT. Additional chemical attachment strategies have been developed, some are on the external surface, others inside the hollow form. Extreme effectors use the CNT scaffolding as a sacrificial structure to be removed by heat or chemical treatments leaving another structure that would be difficult to fabricate without the CNT scaffolding.

DNA coils around the external surfaces, an important part of delivery to a cell. Smaller diameter CNTs, and voids between concentric CNTs, offer advantages for loading and unloading using capillary action, eliminating any specific end effectors to achieve some functions.

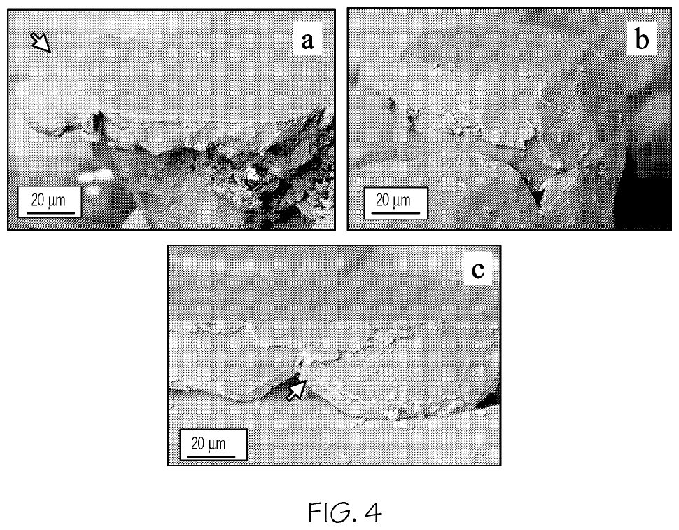

FIG. 4 shows a high-resolution image of a highly polished surface. The nature of metal polishing has been a central part of optics designs for centuries. Lensing with shaping has become a more popular optical solution as surface roughness and aging of surfaces reduce image quality for scientific measurements. Asperities, micron class, will be a major obstacle to accelerating nanoparticles in a central force device.

Another factor in asperities considerations will be the large numbers of parallel channels required, thousands per central force accelerator device, which makes conventional polishing a less attractive solution even if the asperities can be made smaller than the nanoparticles.

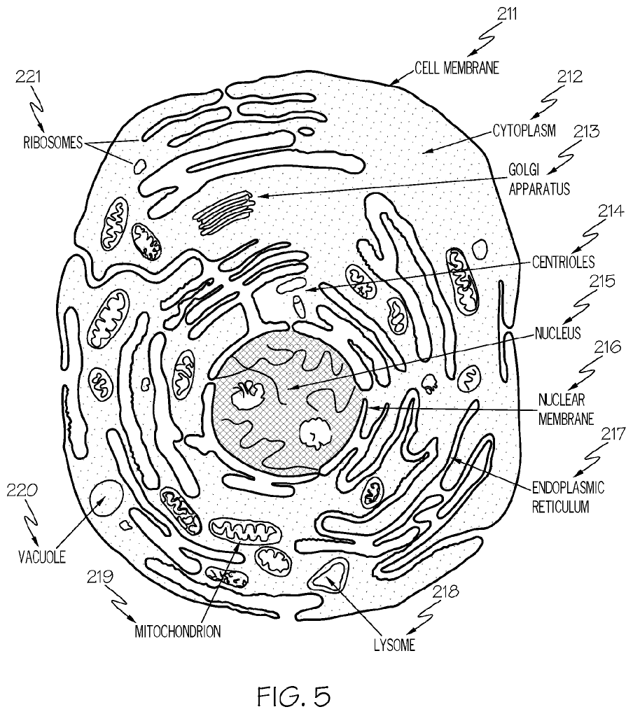

FIG. 5 shows a typical animal cell, with internal components (organelles). Cell sizes vary, but for most applications related to this patent application the average size is 20-30 microns, in each of three dimensions. Biological penetration into the cell's volume requires a chemical ‘key’ to be admitted beyond the cell membrane, object 211, however, in this application the ‘key’ is bypassed by simple kinetic energy (projectile mechanics). Depending upon the ‘target’ location various projectiles' designs will be employed. DNA projectiles, destined for the nucleus, object 215, require more precise targeting as kinetic must also be matched to the penetration of the nuclear membrane, object 216, unless a composite projectile design is used wherein the DNA is able to be moved across the nuclear membrane, object 216, once inside the cell membrane, object 211. All other projectiles are destined for the volume between the cell membrane and the nuclear membrane. Most of the volume is defined as cytoplasm, object 212. Numerous organelles are occupying space within the cytoplasm, these organelles include; Golgi Apparatus, object 213, Centrioles, object 214, Endoplasmic Reticulum, object 217, Lysosome, objects 218, Mitochondrion, object 219, and Ribosomes, object 221. Vacuole, object 220, are potential targets, but their purpose is more interesting in plants.

Which organelle is targeted varies with the projectile purpose. Projectiles with mRNA could target Ribosomes, object 221, whereas cancer drugs could target almost any organelle to stop reproduction.

Functions of each organelle are well documented in textbooks. However, as complex experimentation is accomplished using the penetration strategy defined in this patent application new knowledge is potentially gained. Hybrid experiments could be expedited with larger number of tests, on larger number of targets, at a faster pace when compared to current testing protocols.

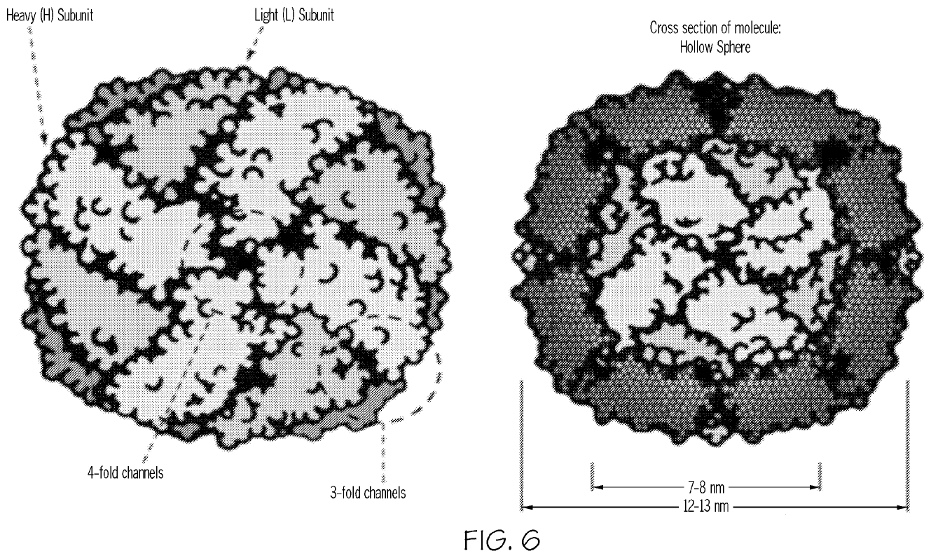

FIG. 6 shows a Ferritin, color images provide a richness hard to appreciate in black shades. Ferritins are useful as transport containers, their nature function in biological systems, the Heavy (H) Subunit, and Light (L) Subunit, and 4-fold channels, and 3-fold channels are terms used to describe the proteins structures. Apoferritin is produced from Ferritins through chemical processes, and have been developed into generic host carriers, in the 7-8 nm Hollow Sphere defined by the folded proteins.

The near spherical shape of Ferritins gives them a low resistance to motion inside a central force accelerator structure.

Apoferritin is another example of a nanoparticle with potential application as a projectile. Readily available, easily converted into a transport system, and requiring no medical certification because they are abundant in living organisms including humans.

Artificial carriers, projectiles made from other proteins are also practical carriers.

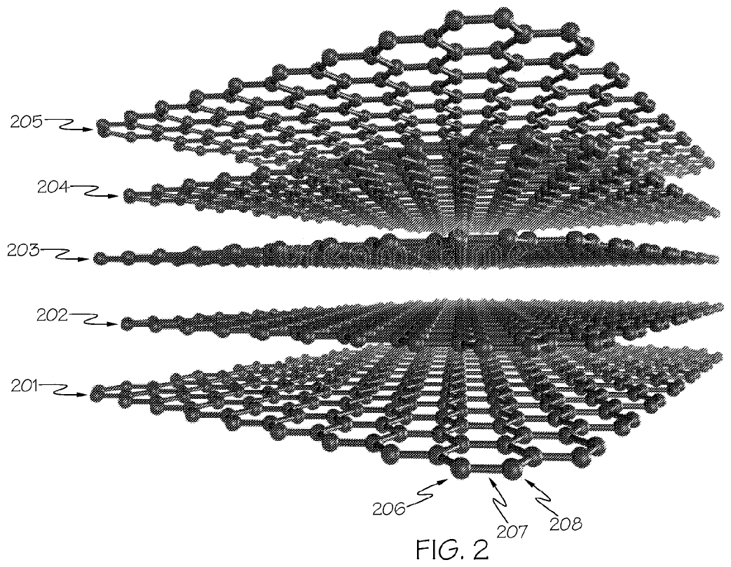

FIGS. 1-3 are mechanical aspects of interesting candidate materials, a series of mechanical images of nanostructures for carbon; FIG. 1 triple walled carbon nanotubes. FIG. 2 shows sheets of Graphene. FIG. 3 shows some typical attachments for carbon nanotubes including surfactants. FIG. 3 shows an external CNT surface mechanical structure. FIG. 4 shows a polished surface reflecting the dimensional values of asperities.

Two images: a classic animal cell representation, FIG. 5, and a Ferritin molecule in FIG. 6 represent the complexities of cells, and smaller Ferritin objects that interact with cells.

Size is critical to these selected images. The cell, FIG. 5, is about 30 microns in each dimension. In the polished surface image in FIG. 4, the scale reflects roughness at the 20 microns range. CNTs diameters are as small as several nanometers, and attachments, FIG. 1-3, are molecular. Graphene, shown in FIG. 2, has a surface roughness at the molecular scale, the third dimension, that is mostly ignored. Ferritins sizing is noted on FIG. 3, at about 13 nanometers external diameter with an internal void of 7-8 nanometers in diameter in Apoferritin.

The triple walled Multi-Walled Carbon-Nanotubes (MWCNTs), FIG. 1, is typical of the structures commonly fabricated. Dimensional values for CNT diameter and length can vary, techniques are established to sort the MWCNT, or even Single Wall Carbon Nanotube (SWCNTs). The surface roughness and spacing between layers in MWCNTs has utility for accelerating nanoscopic projectiles.

Of particular interest is the repetitive nature of the bonding structures for CNTs and Graphene. The most significant aspect is the relationship of CNTs to the Graphene structure; conceptually CNTs are sheet Graphene rolled into a tube. CNTs and Graphene have intriguing mechanical and electrical properties. Many other materials have properties similar to carbon's CNTs and planar sheet of Graphene. Material Science teams have made numerous configurations of non-carbon structures that have properties that are useful, one specific example is Boron-Nitride.

FIG. 4 has asperities that are too large for nanostructures to be accelerated across under a central force. Polishing of non-optical surfaces is not sufficiently developed to eliminate the asperities. While 20 microns is adequate for flowing fluids of quantities vastly larger than one cell's volume the coarseness will not work for nanoparticles defined as a sub-cellular volume.

FIG. 5, a typical animal cell, shows the classic sub-cellular components. External cell membrane is defined, as are many intra-cellular components. The nucleus is the only component that contains DNA material. Altering DNA is a critical aspect of the invention, whether it be in an animal cell or a plant cell, or microbe, or hybrid plant-animal.

Within the cell membrane boundary, object 211 of FIG. 5, but outside the nucleus, object 215 of FIG. 5, are many organelles. Targeting any of the specific functions performed by one or more organelles outside the nucleus can be beneficial. Treatments of cellular malfunctions such as cancer, could be accomplished by stopping the cell's reproduction or even its viability by poisoning the cancer cell.

Ferritin, shown in FIG. 6, is a specific structure made of proteins, nominally associated with iron movement within an organism. However, Ferritins can be carriers of many different molecules or compounds. Processes to allow Ferritins to act as universal transport tools have been created for a vast array of objects to be carried into a cell and released for some specific function. Cell membrane penetration of Ferritins is part of normal biological functions. Entry into cells via the circulatory system is the convention used by most applications of Ferritins. This patent application does not require the circulatory system for access to the cell, however, using a carrier that is naturally found in cellular activities reduces the variables in functionality of the biological actions.

Combinations of usage of these nanoparticles, either as a transport vehicle, or as a nanometer smooth surface within the central force accelerator, is illustrated by these examples.

Accelerator structures could be MWCNTs wherein the spaces between tubes, or Graphene layers, acts as the surface allowing other nanoparticles to move unimpeded along the accelerator's pathway from insertion to exit. Likewise, other nanoparticles, Boron Nitride nanotubes, could be used as the accelerator pathway. Or gold plated, atomic scale, on MWCNTs, or other structures.

The richness of possible nanostructures for the pathway structures, and projectiles, is subject to new techniques being developed each year in various research facilities.

The critical takeaway from these drawings is that the technologies exist and can be employed with a central force acceleration concept to launch nanoparticle projectiles directly into cells without any other biological support infrastructure such as the circulatory system.

This application defines components and integrated systems using; (1) specialized nanometer-class projectiles, (2) nanometer-class metering devices, (3) central force accelerators, and (4) targeting strategies, to establish a credible solution to advancing the State-of-Art for cellular medicine development, and to treat specific illness and disease in humans. Treat each cell as if it were an isolated self-sustaining entity, for a short period of time, for the purpose of altering its' future state. There are two levels of this altered state; (1) nucleus alteration of the DNA, and (2) augmentation of the composition of the non-nucleus portion of a cell. Alterations are done via projectiles with specific purpose, wherein the projectile is inserted into the target cell by kinetic processes; effectively nanometer sized bullets penetrating until stopping in a cell.

Targeting an individual cell with a nanoscopic projectile, or projectiles, capable of influencing that cell's future behavior, and nothing else, is a novel strategy. For cancer treatments a single cell is nominally not known to need to be targeted as a disease will not have expressed itself. For drug production the targeting of individual cells is a practical means of inducing specific cellular actions. This system is capable of targeting one cell or a cluster of cells.

A mechanical system to accelerate nanoparticles for direct insertion into cells is developed, generating new opportunities for nanoparticles applications in biological processes. Medical usage is one utility, another utility of nanoparticles is in materials development using biological ‘factories’ coopted to create novel products.

The nanoparticles are subjected to a central force mechanical acceleration profile. A central force configuration fills a niche between indirect insertion of nanoparticles into cells using circulatory processes, and direct insertion Electromagnetic (EM) devices capable of handling only the smallest masses (protons and electrons).

The nanoparticles are known and used in medicine, intravenously. However, limitations are imposed as nanoparticles' complete side effects are somewhat unknown as they interact with tissues which were not the target tissue. With more precise targeting provided for in this application the risks are more confined to very localized regions. Additionally, nanoparticles previously not deemed medically useful or medically allowed due to toxicity, could become medically useful. Correcting human genetic induced health problems, such as birth defects, occurs as cells are targeted with nanoparticles hosting the genetic correction; either within the cell's cytoplasm Ribonucleic Acid (RNA) or nucleus Deoxyribonucleic Acid (DNA).

Manufacturing also has potential usage of nanoparticles to change the processes used in fabrication of medicines and materials. Complex plant, or animal, or plant-animal, genetic interoperability enables and enhances material science research by using nanoparticles to transport raw ingredients and/or genetic content to cells within an organism or modified organism. Researchers are enabling many more amino acid combinations, to be biologically formed into proteins. These protein implementations can be enhanced with direct cellular insertion of nanoparticles hosting genetic instruction and/or raw ingredients, by a central force machine.

Three very different mechanical designs for an accelerator device (the central force implementation tool) are considered; gyration based, spin-based, and combined spin-gyrate. These have a few common features; nanometer class surface smoothness for the accelerator surface transferring momentum to the projectiles, and large numbers of individual acceleration surfaces (aka pathways or channels), pathway-to-pathway separation that matches the nominal target cell spacing of 30 microns. Achieving sufficient momentum to penetrate a plant cell membrane, and outer cell wall, is not difficult, however the penetration of tens, hundreds, or a few thousand cell membranes in a human will be challenging. Regardless of the momentum requirements there are physical barriers to accelerating very small objects; mostly the small surface area of mechanical interaction to transfer forces from the central force surface to small nanoscopic projectiles; make the projectile move inside an accelerator along the pathway from the entrance portal to the exit portal.

The components of a medical device are; (1) source of nanoparticles with defined purpose, (2) a gating system capable of metering small quantities of nanoparticle projectiles into one of many acceleration channels at the frequency of the targeting opportunity, (3) massively parallel acceleration channels, matching the target cell spacing, (4) each channel's surface asperities are smaller than the projectile to prevent trapping projectiles inside the channels, and (5) cellular targeting capacity to support meaningful treatment in an hour or less.

Projectiles are defined in general terms. There are many options to capture all the work. Many future projectile designs are anticipated. Products are available today, they can be used as is, some might be modified. Ideally medical products such as cancer drugs can be immediately used without new approval due to the delivery system (a mass accelerator direct delivery to the cell of interest versus a circulatory process of delivering a drug to the cell). Industrial systems will probably develop many new projectile types as the processes they use are more flexible, their end-state products are the controlled items.

Metering, per channel, is very critical. Extremely small mass/volumes flow into a specific channel each cycle of the system. Gates capable of restricting a ten nanometers object, and operating at hundreds of hertz, are baselined. Additionally, there are hundreds to thousands of gates required for a system to function. Gates will most likely be fabricated using nanoparticles and high-performance piezo electrics. As a goal the mass being metered could range from a few thousand nanoparticles to over one-hundred thousand per cycle, per channel. As a reference a typical 30 microns cube would hold about ten billion nanoparticles of the Ferritin-class. Inserting a mass/volume that is more than can be accepted into a cell is not useful unless the goal is to burst the cell.

Thousands of channels will be operating each cycle of the system, system cycle rates could be 200 hertz, or more. Some piezoelectric options are in the kilohertz range. Channels' cross-sections are tiny when compared to target cells cross-section. The channels require a sophisticated surface quality. Achieving the surface quality can most easily be accomplished with nanoparticles, specifically sheets or tubes with atomic roughness (no real asperities). Channels asperities, when at the atomic scale, are also over short (atom-to-atom) spacing of about 0.1 nanometer, whereas the MWCNT or Ferritin projectiles are several nanometers, to tens of nanometers. Thus, a CNT is a ‘long object’ resting on many atoms of the accelerating surface, hence very good contact to allow transfer of momentum between the accelerating surface and the projectile.

Medical benefits must be timely, thus the system needs to address quantities in a reasonable period of time, and the same is true of manufacturing functions. It is reasonable to assume any medical treatment is done at a facility hosting specialized equipment, including devices made using this patent application. Millions to maybe a billion target cells are possible when treating cancer, or adding mRNA to lung cells, or any organs' cells. Performing those actions in 1,000 seconds or even an hour (3,600 second) at high projectile repetition rates of 200 hertz requires up to several thousand parallel acceleration channels. These numbers are used as preliminary system requirements, with some flexibility in them. However, these raw values are achievable with technologies available today; there are no ‘magic events’ required to build numerous versions of machinery to accomplish the objectives of this patent application.

Much of the design work will come down to a few technologies; nanometer class materials used in projectiles, metering the projectiles into the acceleration devices, and more nanomaterial used to provide a low asperities surface in the acceleration zones, piezoelectric actuators used in metering and potentially providing motion to the central force that accelerates the projectiles (for gyration designs), and a host of projectiles' processes (Ferritins and MWCNT). Magnetic bearing could be used to achieve both surface contact friction and motion of the surfaces relative to each other; the rotation and gyration motions can be generated by electro-magnetic forces.

Coupling these nanomaterials, piezoelectric, and acceleration structures is a novel way to provide direct penetration into cells without using the circulatory system, and to address fundamental research capabilities, and subsequent production of material based upon biological factories replacing older techniques.

Different classes of targets also are known; small angle of dispersion targets such as cancer tumors, and wide angle of dispersion applications where plants are being sprayed. Simple gyration designs, single frequency and fixed gyration radius and no rephasing, are more favorable for the small dispersion applications. Rotational designs, simple tube rotating at a fixed rotation rate, are more suited for spraying applications. Another class of gyration design is complex gyration; multiple stages each with a stage unique frequency and a stage unique gyration radius, and all stages can have rephasing. Complex gyration designs are compact units, and can be easily handheld, more portable than the rotating design for the spraying wide-angle dispersion. The exit velocity is lower for plants than human targets as the penetration depth, number of cell layers, is one or two for plants, and potentially a thousand cell layers for human tumors.

### Projectiles:

The projectiles are defined as nanometer-class, with purpose defined by their application. Molecules (CNT) and compounds (Ferritins) can be projectiles, and each is capable of hosting other molecules or compounds. Specific shapes, composition, and sizes of projectiles are acknowledged in broad ranges. Some projectiles are more characterized than others, and others will be newly created as utility becomes known.

Nanoparticles projectiles can include any compound, especially water-soluble compounds, or compounds made into colloidal suspensions. Radiological and chemotherapy compounds can be either water-soluble or colloidal suspensions. Drugs, based upon nanotechnology, with other medicinal purposes, are also used. With the exclusion of sub-atomic particles, most, if not all, medicines currently in use can be processed into nanoparticle projectiles.

Individual nanoparticle projectiles are smaller than a cell (nominally a billion times smaller by volume), and likewise smaller than cell organelles (approximately several hundred thousand to one million times smaller by volume), they will experience more organelle interactions than protons and electrons from an EM accelerator. The nanometer projectiles could have hundreds to millions of electrons and protons. What happens when a nanoparticle interacts with an organelle depends upon the collision mechanics.

Three collision types will occur in most nanoparticle projectile motion through an animal where many cell layers are involved. Initially the projectiles will interact with organism's outer layers of cell-like structure (human skin being a prime example), there are no living functions in dead skin cells. The second category of collisions is a first living cell's membrane, with more collisions occurring until the last collision, and there might be thousands of collisions, with semi-rigid cell boundaries. The third collision type is with organelles within any cell between the dead skin and the ultimate target cells' cytoplasm. At the cell membrane the nanoparticle projectile will either pierce the membrane, or not. When the nanoparticle is attempting to penetrate into or out of a cell, the cell membrane's localized composition will define the energy (momentum) required.

Superficially the collision within a cell is between two objects in a fluid, where anchorage is essentially non-existent, thus collisions will cause revectoring of the interacting particles. Classic physics might suggest this is Brownian-like motion.

A last collision occurs when the nanoparticle is lacking momentum sufficient to penetrate the cell membrane to exit the cell, the nanoparticle is trapped in that cell. Once trapped the nanoparticle has opportunities to further random interactions, with other constituents within the cytoplasm; this is where the specific designed functionality of the nanoparticle's purpose is reached.

Two fundamental nanoparticle projectile shapes are likely to dominate the system designs; spherical and cylindrical. Spherical Ferritins, a biological compound, are known to host many molecules and compounds, with usage in various industries and medicine. Cylindrical CNTs are ubiquitous, used as medicine too. Nano-aluminum, nominally spherical, is a medicine, dissolved and used intravenously; side effects on non-targeted cells are a concern. Nanotube compounds and near two-dimensional compounds, Graphene-like, are developed with various processes and purposes, yielding different products. Researchers have doped carbon-based nanotubes and Graphene with many combinations of elements using unique processes, amazing results have ensued. Abundant nanoparticle compounds are known and all are potential projectiles, some are actual medical treatments. Projectile nanoparticles have scaled up versions of the same materials, with another usage in the structures of the channels, used in the acceleration of projectiles. Other versions of these scaled-up nanoparticles are useful in metering nanoparticles into small mass/volume units.

Cell volumes and organelle dimensions are an important part of selecting the quantity of projectiles to use, and what configuration these projectiles should take. There are target cells, where the projectiles are intended to stop and execute their functions. Collateral damage cells are between the central force accelerator's exit portals (plural) of channels and the target cells. Nanometer-class projectiles are favored over larger projectiles, smaller cross-sections are highly desired to reduce collateral damage, recognizing the cross-section of a typical cell is 30 microns by 30 microns. Spherical Ferritins are about 15 nanometers in diameter, one-four-millionth of the typical cell's cross-section. Nanotubes molecules made from carbon, and nanotube compounds such as doped CNT and MWCNT, have selectable dimensions. It is very realistic to assume the cylinder diameter of 5-30 nanometers, one-millionth the cell's cross-section or less. Ferritins are defined volumes, but nanotubes can be of variable length and diameter, thus variable volumes. Longer nanotubes, with diameters equal to or less than 15 nanometers can host more ‘payload’ than a fixed Ferritin sphere, and these much longer nanotubes can be imparting larger momentum due to their larger mass. Momentum defines the penetration depth.

Any one projectile's content (payload) may be a part of a total mass delivery to the target cell. Projectiles could be clustered as part of the total delivery. Different shaped nanoparticles can be grouped to take advantage of collision dynamics and fluid dynamics as projectiles pass through cells located between the accelerator's exit portal and the target cell.

Projectiles are formed from; (1) compounds shaped into nanoscopic form, (2) compounds that are naturally nanoscopic such as Ferritins or molecular CNTs, or combinations of the naturally occurring nanoscopic carriers (Ferritins or CNTs) hosting a cargo of some other compound or molecule. Cargo carrier nanoparticles are well established technologies.

No specific projectile is being claimed, but it is obvious the subcellular sizing of projectiles is unique to this design strategy. Advances in development of central force accelerator solutions, specific for nanoparticles, will foster new compound designs and form factors.

Building a specific nanoscopic projectile is non-trivial simply due to the extremely small dimensions. However, Ferritins are predefined, with an existing loading process. Nanotubes are also sized according to length and diameter, and loading has been developed. Specific compounds or molecules to be loaded, into or on carrier compound or molecules, have not been fully defined.

Unloading Ferritins is well established. Unloading nanotubes has also been done for some molecular nanotubes and nanotube compounds. As additional nanotube structures are developed new loading and unloading techniques will be defined.

What loading and unloading processes are employed for specific applications is unknown, but sufficient methods are available for individual users to select from a menu with their unique needs being the deciding factors. This menu will be expanded as research teams develop more nanoparticles.

However, for a central force accelerator design team it is important to support all the various aspects of any projectile selection. One obvious solution that potentially services all design needs is a massively parallel accelerator structure hosting thousands of channels and allowing the user to select which channels are actively used. Moving projectiles into a channel from a supply system, after metering the desired mass/volume of nanoparticle projectiles is essential.

Applications will define the nanoparticle designs. For pharmaceuticals the plant and microbe chemistry will be unique to the desired end-product. Cell sizes and overall thickness will also influence the nanoparticles, wherein momentum is smaller as depth of penetration is shallower.

When addressing animal, and more specifically human trials, the risks of an undesired outcome will factor into nanoparticle designs. This situation is like any other drug trial. A novel delivery and drug and/or genetic material formulation is involved. A serious starting point for human injection strategies is to address existing approved drugs, and adapting the dosage to be a projectile rather than a circulatory system delivery. In principle the drug is already approved for the purpose, and Needle Free Injection is known (just not in this particular configuration).

Nanoscopic projectiles are created by selecting a compound and forming a small collection of molecules into a shape. By example, some common cancer drugs are; trans-retinoic acid, arsenic trioxide, asparaginase, eribulin, ixabepilone, mitotane, to name a few. Arsenic trioxide has molecular dimensions of a few Angstroms, and forms chains. Trans-retinoic acids are larger molecules, but still measured in Angstroms. Ten (10) Angstroms are equal to one (1) nanometer. In this application the projectile sizes are at least 1 nanometer in extent in each axis, and more often projectiles are tens of nanometers per axis. The largest projectiles will be a few hundred nanometers on at least one axis, and maybe all three axes. The absolute size of any projectile is selected based upon many parameters, including the depth of penetration.

Deeper penetration requires more momentum, which can be achieved by increasing the mass or velocity or both. Another parameter for determining the size of any specific projectile is damage done to healthy cells as the projectile transits through the healthy cell enroute to the cancer cell. Cell-to-cell spacing is about 30 microns for most human cells of interest, so even the largest anticipated projectiles are smaller than the cells being targeted. Ideally the projectile's cross section is small compared to cell dimensions to increase the probability of full penetration without impacting some internal organelle that was not targeted.

If the compound is solid, and capable of retaining its form under radial acceleration of the central force machinery, then the nanoscopic projectile can be finalized by making small molds and filling the molds; similar to making any other solid object, some solids are held with a binder material. Small molds are potentially made from cast using nanotubes as the framework or skeleton.

Many nanotubes are also commonly fabricated, boron-nitrate and silicon-based structures are well established. Silicon nanotubes have had minor success at killing cancer cells, reference article is found in Nanotechnology and Functional Materials for Engineers, 2017. However, these experiments lack a delivery system.

If the compound is solid and not capable of retaining its form under the radial acceleration of the central force machinery, then the compound can be encapsulated in a cage molecule, such as a Ferritin or CNT. In some cases, the compound will be encapsulated because the projectile form is best served by the form of the encapsulating structure; CNT can be selected to have a specific length for a given diameter making them more likely to pierce the cells between the exit from the central force machinery and the target cancer cell region. Conversely, Ferritins are spherical in shape and this shape may be advantageous in some deposition strategies as the projectile's cross-section is uniform from all aspects. Combinations of different projectile form factors may best serve the specific treatment.

Compounds currently not approved for cancer treatments are potentially useful as the projectile nature of the treatment eliminates conventional risks to the patient generated by circulatory system distribution. Some of the compounds are known drugs, others are not considered drugs. Most metals are toxic at some concentration, at a cellular level these concentrations can be achieved without poisoning the whole person. Oxidation is typically an exothermal reaction, and these can be exploited to locally heat a cell. Endothermal reaction can be as effective as exothermic. Opening up the options to all chemical reactions creates many potential projectile constituents, including complex compounds delivered in separate projectiles and reacting after delivery into the cancer cell. Making a cell burst, due to internal pressure is another selective killing strategy.

Genetic disruption is a tool for killing cancer cells using projectiles to affect the genetic processes within targeted cells and not elsewhere in the body. Without use of the circulatory system distribution no other cells' genetic composition is being altered. Some practical genetic disruption techniques are insertion of genetic materials into the cytoplasm altering the genetic instructions for cellular operations. These instruction-based corruptions can be at any stage of cellular actions, so long as the end result is the termination of cellular reproduction and death to the cancer cell, and eventually all the cancer cells are killed. Another practical disruption of genetic processes in the cancer cell cytoplasm can occur with a chemical that prevents one or more processes from having sufficient raw stocks to complete a cycle; bind up a receptor site on a segment of genetic instructions is an example. Altering the cancer cell DNA is possible, but requires targeting the nucleus, which is more challenging than targeting cells in general.

### Projectiles' Metering;

Metering nanoparticles insertion into the central force accelerator's pathway from a supply container has a multitude of options. Solid and liquid nanoparticles are easily moved by simple pressurized gases. Furthermore, the concentration can be managed with simple mechanical mass sorting equipment. CNT end-effectors can be electro-magnetic, or sensitive to pressure (or lack of pressure). End-effectors are control agents.

FIG. 3 shows how various attachments can be added to nanostructures, and those attachments can be used to act as metering tools. Additionally, metering can be electro-mechanical using forces from EM fields to cause distortions which either allow or disallow movements within a volume. Another metering strategy can be accomplished with pressure gradients, positive pressure zones push nanoparticles into the central force acceleration channels, and negative local pressure holds off nanoparticles from entering the central force acceleration channels. Persons skilled in these fields of research, including effectors on nanoparticles, have developed many nanodevices for different purposes, and the control of their movements under external forces such as electro-magnetic and/or pressurization.

Nanoparticles themselves can become measuring containers, with open and closed portals much like a set of locks on a canal shift water to move boats. Very large diameter doped CNTs are possible measurement containers. Projectiles' quantities are delivered to the entrance of a channel, one of hundreds to thousands of channels, for acceleration under the influence of the central force. These channels are refreshed at some duty cycle, with some repetitions being at 200 hertz or higher.

The mass, or volume if that is the metric of measurement, of each set of projectiles entering any one of many pathways, within the central force machinery, is an engineering design option. Suffice it to say the options will expand as different end-effectors are created.

Simple mechanical chopping, making moving structures with openings moving at different rates, easily operate at 200 hertz or higher rates, piezoelectric devices operate over a broad range, few hertz to ten kilohertz or higher. The projectiles advance to the next stage when multiple openings coincide, the final stage for any projectile is the entrance to the central force accelerator's channel.

Ferritins can be monitored by adding magnetic content inside the volume. Spherical shapes can be moved by other forces, before entering the central force machinery. CNTs can be guides to select Ferritins. Even as nanometer class objects these can be controlled by pressurization forces. Ferritins by nature will move by rolling. Many filtering, or quasi-counting techniques, are known to work on fixed volumes and Ferritins are just balls that fill a void to capacity, then go into overflow basins. 100,000 Ferritins have a mass of less than one picogram. The mass of several thousand channels' nanoparticles is in the nanogram range. While small it is quantified and can be mechanically separated from other masses.

Automated nanoparticle separation systems will be used, the exact fashion will become a design detail when a utility is specified. Of specific note is the flow rate per cycle, which is thousands of metered quantities per second, thus initial masses are micrograms to be subdivided each cycle into nanograms, and then fractional nanograms.

One interesting feature of nanotubes is the secondary Van Der Walls forces found in the relationships between nanotubes and the compound inside the nanotubes. This force can be an indirect tool to assist in metering projectiles into the accelerator.

CNTs hold unique electric properties allowing for gating at electrical rates. The chopping function mentioned above could be an electro-mechanical system. Graphene sheets also express interesting electrical properties.

Piezo-electric actuators can be positioned to make structures shift the size of openings, valves at a nanometer scale, again these operate at kilohertz rates, much faster than the nominal 200 hertz of a rotating mechanical shaft driving the central force accelerator functions. Graphene sheets can be positioned in stages, with piezo-electric actuators. Sheet-to-sheet spacing is selected to match one array dimension, with cell separations between individual sheets. Twenty-one Graphene sheets pairs (total of 62 Graphene sheets) are spaced 30 microns (cell spacing) apart gives twenty feeds (gaps between any two Graphene sheets). The output of these twenty flow restrictors feed a second set of pairs, providing the second dimensional restrictions for the two-dimensional central force accelerator array. Pairs of piezo-electric open and close to form defined voids with defined volumes, and by default defined quantities of nanoparticles, at whatever rate is needed (these devices operate at rates much faster than required for the channel feeds).

When the pathways are defined, most are two dimensional arrays, the feed mechanism will need to match the array composition and relative spacings.

In the designs of the multi-stage gyration systems, there are stage-to-stage transfers. These designs are patented by the inventors of this application. As a function of projectile velocity, during a stage-to-stage transfer a gate can allow, or restrict, flows moving from a slower stage to a faster stage. A filtering network can be accomplished in a similar fashion between the supply feed and the entrance to a series of two-dimensional array elements.

Many options exist to provide proper insertion of nanoparticles into the appropriate array elements of the central force accelerator. Small quantities of projectiles are being used in each cycle of each channel, from a few to 100,000 projectiles destined for a specific target cell, as opposed to a large number of projectiles, such as one whole cell's volume of projectiles (10¹⁰—crude estimate), means some metering system is required.

Larger numbers of nanoparticles can be gated, this is a medical decision, supported by design parameters.

### Central Force Pathways:

Nanoparticles also are useful in making the pathways, the many channels required in the designs. Thousands of parallel channels are required to address meaningful quantities of cells in a typical treatment duration for a cancer patient.

By example a simple gyration solution, addressing a grape-sized tumor (100 million cancer cells) need over 1,000 parallel channels sending projectiles into the cancer node over a 15-minute period, wherein the gyration frequency is 200 hertz. Acceptance of the number of channels is important to the pathways being designed into an accelerator system.

Nanometer class projectiles require an acceleration pathway that is much smoother than any conventional polished surface. As noted in FIG. 4 conventional polished metal has asperities in the 20 microns range; essentially a mountain range compared to the nanometer class projectiles. If the smoothness is typical medical grade smoothness (microns), then the asperities will trap the small number of nanoparticles; 100,000 nanoparticles occupy a very small volume, smaller than the traps of 20 microns class asperities, and there would be asperities all along a polished metal surface.

Nanotubes (compounds more than molecular carbon) have been created to be macroscopic in length, while being nanoscopic in cross-section.

In FIG. 1, Multi-Walled Carbon Nanotube (MWCNT), are three surfaces with atomic scale smoothness. Each tube's inner wall, tubes 101 and 102 and 103, could be the surface a nanoparticle projectile moves along as the central force adds velocity to the projectile. Selecting a spacing between tubes also helps keep the nanoparticle from losing velocity due to random collisions with rough surface, as found in polished metal. All the nanoparticles are bigger than the spacing between bonded atoms in the CNT as shown in FIG. 1.

Another aspect of the nanotubes is spacing between them, which can be another channel. Projectiles are going to move in a deterministic way once under the central force acceleration, so projectiles can be managed by how they are staged prior to entering the acceleration zone. Different components of a target cell's nanoparticle projectile suite could be selectively accelerated in different parts of a MWCNT.

The very materials used in making projectiles offer a surface that can be used for the acceleration pathways. Graphene is smooth to less than 1 nanometer. MWCNT's has voids between the concentric tubes that are also smooth to less than 1 nanometer. Numerous teams have created very long MWCNTs, sufficient to be the complete pathway.

Another strategy is to place many MWCNT in close proximity and use the spaces between the external surfaces as the void wherein different projectiles transit the acceleration under the central force.

Various doped CNTs are also candidate channels, the same materials used as metering devices.

Sheets of Graphene can be used where a void between sheets is the acceleration zone, and the projectiles are bound by a curved set of layered Graphene. A full rolling of Graphene becomes a carbon nanotube. If Graphene sheets provide surfaces, and many layers of Graphene are built into a composite, then the spacing, potentially modulated by piezoelectric actuators, will act as individual channels for projectiles.

There are several ways to build any array, including the 20×70 elements used as an example. One array element (30 microns by 30 microns) is a cluster of 1 million MWCNTs (30 nanometers in diameter) wherein different channels can be activated. There are 1,400 elements in the overall array, thus 1.4 billion MWCNTs (a large number), and each is made to be a full pathway's length, potentially a meter or more in length. Commercial production of MWCNTs in these quantities and length are the SOA. Once a demand exists for these as fibers in fabrics production will expand. Alternative fabrication is a smaller number of MWCNTs, with the remaining volume in the array elements being filled with another material. Yet another fabrication technique is to cut channels into a thin foil, use piezoelectric to control the contour, many foils would be stacked to provide an array format. These channels would only need one surface contoured, that surface where the nanoparticles contact the channel as the channel transfers momentum to the projectiles in that channel.

Between the MWCNT are structures to house the MWCNT and maintain the relative spacing. Since the shape of the pathway is potentially a spiral, other shapes can be made to work, as references in gyration patents. Metal form, strongback material, could house the array with additional filler materials such as glues or elastomers. Numerous materials are available, and a designer will select from the many options. Persons with mechanical engineering skills will select the filler based upon the acceleration profiles expected (loads).

### Accelerators:

Describing the machinery, in general terms, is dependent upon the acceleration technique. Three techniques of central force are patented by these inventors and will be the basis for designs outlined in the applications sections. Other acceleration techniques are in consideration.

Within the pure gyrate constructs are several themes, a single phasing system with fixed parameters for diameter, gyration radius and gyration frequency, a multiple phasing system wherein the gyration phase is reset during transit, gyration radius and gyration frequency are potentially variables. Pure spinning acceleration has two parameters, diameter and frequency. Complex spin-gyrate has three parameters; diameter, phase relationship between the spin segment and the gyrate segment, and frequency.

Directional control comes in several varieties as well. One consequence of gyrating structures is the exit portal is more or less fixed, and with simple deconvolution of the motion the exit can be made fixed. However, as the target region will nominally require motion in the axes perpendicular to the projectiles' direction of motion the natural motion of gyration may be selected to match the area coverage aspect of a treatment.

Spinning motion has more complex control requirements, but very doable. Electronic gating of the exit is simple, and at high revolution per minute designs for a 1-2 meters rotating structure, hosting tens of thousands of channels, the use of microprocessors to select the output position around the 360 degrees rotation is straightforward. Persons skilled in electronic gate controls on engines and other rotating machinery have solved this problem many times,

Discontinuities within a pathway, as found in spin-gyrate and multiple phasing, pose complexities can be overcome with larger diameters or higher frequencies for the other options lacking the discontinuity in pathway. Persons skilled in the arts of spinning and gyration machinery can adjust the parameters to achieve a wide range of momentum results for any specific projectile design (as defined by the individual projectile's mass).

A billion cancer cells, of 30 microns average dimension per axis per cell, is a cube 3 centimeters on each axis (a large tumor). In another parametric, the relative pathway separation of 30 microns the concepts of gyration, at 200 hertz, and a treatment window of 60 minutes (3,600 seconds) requires approximately 1,400 parallel pathways, assuming one projectile set per target cell, if 1 billion cells are the target cluster. 1,400 could be an array of 70 elements in one dimension (‘X’) and 20 elements in the perpendicular dimension (‘Y’). At 30 microns per element the 70 elements are 2,100 microns (2.1 millimeters). The whole array is 2.1 millimeters by 0.6 millimeters; quite small. A scanning process would move the array in the X-Y dimensions, while momentum is controlled by changing the gyration frequency around the 200 hertz. Depending upon the volume (shape) of the target cluster of cells will determine the required array translation requirements. In conjunction the depth of target cells will require momentum changes to reflect variable depths of penetration. Numerous target acquisition and fire control designs are available to achieve those functions. These electro-mechanical design considerations are typical for any complex machinery, and persons skilled in the art of deconvolving requirements into specific designs will find a range of acceptable answers. This treatment could be scheduled over several windows to kill all the cancer cells.

Precision opens opportunities for treatments, research, and eventual production of novel products. Different central force accelerator designs apply to different cellular targets, mostly defined by the penetration depth and the angular dispersion requirements.

For all gyration-based designs the exit is very restricted to just the angles defined by the tip motion related to gyration motion deconvolved from a circle to two one-axis translations. One of the two one-axis is in the direction of projectile flight, the other is perpendicular. The perpendicular axis is a translation motion that can be coupled into desired translation coverage, or the motion can be fully compensated with a secondary structure, all of these are defined in various gyration patents. The net effect is the projectile pathways after exiting the acceleration pathway of the central force machine is deterministic.

### Medicine:

Providing medicines, as projectiles, to million or billions of human cells means the number of channels will need to be several thousand, and the treatment is at least 15 minutes, and the machinery runs at high Revolutions per Minute (RPM). Assume delivery occurs over a treatment period (15 minutes), with medicines in parallel channels (2,000 is a good starting estimate), trajectories to individual cells. The cycle time for a single delivery is a small fraction of a second (gyration or rotational cycle). Counting the powers-of-ten gets to a billion opportunities. 15 minutes is 900 seconds (call it 1,000 or 3 Orders-of-Magnitude). 2,000 channels are another 3 Orders-of-Magnitude. Realistically the rotation rate is 100-200 hertz, 2 Orders-of-Magnitude. Playing with numbers suggests a billion targets are possible/practical. These thousands of channels need to be fixed in relative spacing to match the target cells. All of these attributes can be designed into several classes of central force accelerators.

Direct nanoparticle insertion, using a central force accelerator, into the cells of interest will have no immediate whole organism consequences, but will have waste products. Any compound can be a nanoparticle; individual molecules are nominally smaller than a few nanometers.

Dose issues will be addressed by multiple nanoparticles. Direct cell insertion has concentration advantages over indirect cell insertion as all of the nanoparticles defined for the cell are delivered to the cell, none is lost to other cells in the body. The only exceptions are projectiles revectored by a collision, or those that find their way into a blood vessel.

Treating a human will require deeper penetration when compared to altering a biochemical process in a leaf on a plant. Likewise, a human is a small angle target whereas a greenhouse of plants' leaves could cover a large solid angle. Different central force accelerator solutions will be defined by the different scenarios and applications. Human target cells can be thousands of cell layers deep into the human. At 30 microns per cell layer, a 12 centimeters deep target is 4,000 cell layers deep.

A leaf has only one cell or few cells in thickness, thus the target cells are in cross section rather than depth. Microbes are more like leaves in terms of thickness. The consequences of these cellular thickness differences have design parameter implications for the central force accelerators used in these different applications.

Absolute size is yet another factor, microbes are small, and need to be ‘processed’ faster in some assembly line fashion, as part of using them as drug product factories. The medium the microbes are housed in becomes the pseudo-target, and the microbes are in effect scanned by projectiles.

Plant and microbe central force designed projectile accelerators have human shallow depth correlations, especially when treating skin diseases.

Direct insertion of nanoparticles into living organisms opens up concepts in medical treatments, and pharmaceutical research/developments, and potentially into hybrid organism research. The exact nature of any specific activity is not being asserted, just postulated. Medical treatments can include augmentations to existing deficiencies in a patient's conditions (more than taking a pill and less than surgery), or new drugs usage for treating a known disease but the desired drug is not acceptable for circulatory usage due to side effects on healthy cells, or genetic treatments at the mRNA or tRNA or DNA levels. Another extreme consideration for medical care could be inclusion of non-human genetic materials to create another symbiotic relationship to foster a higher quality of living for persons needing some compound on a regular basis. Joint pain due to loss of cartilage, replacement materials could be inserted as nanoparticles.

Cancer cells are killed, with compounds formed into nanometer projectiles or embedded inside or outside the volume of a nanometer projectile, via direct penetration into cancer cells, without utilizing the circulatory system. Direct compound injection, at the cellular level, makes this approach like targeted proton therapy. Additionally, killing cancer cells has new options, compounds can be approved drugs at new doses, or drugs not yet approved and possibly never going to be approved because of their side effects when delivered via the circulatory system, but now may have no side effects as delivery is restricted to targeted cancer cells using projectiles rather than the circulatory system. Cancer cells may be subjected to genetic-induced killing, wherein the cancer cell's reproduction is halted by one or more disruptions in the cancer cell's reproductive processes. A genetic flaw can be installed in the DNA (nucleus penetration) or cytoplasm (cell membrane penetration) to effect changes to mRNA, or tRNA, or rRNA, or a host of organelles.

Building better pharmaceutical chemicals through advances in plant and microbe research could be improved by eliminating complexities of interactions to achieve specific reactions within a cell. Achieving access to cellular functionality has restrictions, these restrictions are overcome by clever chemical steps in wet chemistry systems. When a simple penetration is accomplished the number of wet chemistry steps might be reduced, or new options opened up as compounds are allowed in projectile forms that just can't be included in wet chemistry scenarios.

The ultimate advance is insertion of corrective genetic materials into a cell's nucleus lacking the segment of DNA required for healthy cell functions. An intermediate step is to insert mRNA to create a single reproductive cycle for a protein or proteins production for temporary relief from a flawed genetic sequence.

Genetic insertions are also part of the pharmaceutical strategy using plants and microbes with modified functionality. Creation of new proteins for advancing medicine is a practical option once massive quantities of plant and/or microbe cells can be altered in factory conditions.

Cellular medicine development enhances utility by the direct insertion of projectiles into living ‘production’ lines. One configuration of enhanced utility is to generate billions of cell-projectile interactions wherein the plant's cells are penetrated with projectiles delivering unique functionality to the cytoplasm resulting in an equally unique products to be harvested from the organism's cellular excretions. The plant cellular processes can be augmented with genetic instructions that produce medicinal products as waste, excreted by roots or stems or leaves or seeds or flowers. These excretions are the desired medicinal product or ingredient in a medicine. Projectiles can be used to initially deliver the genetic instructions, and subsequently additional components to the cellular processes, including compounds (amino acids) or more genetic instructions.

In plant-animal hybrid organisms, molecular farming techniques have been expanding to produce animal protein from plants. Two reproduction venues exist, one is a living organism able to reproduce with the animal genetic functionality included in the offspring, the other is an organism that does not carry the genetic traits to the next generation. Benefits are afforded to each reproduction solution. Carrying the genetic addition to future generations means the steps of mass production are included in the new hybrid organism. However, not carrying the trait means repetitive plant-animal combinations are required in each growing cycle. Considering the vastness of combination of hybrid plant-animal genetics the benefits of knowing the complete environmental consequences has advantage over picking a few candidates hybrids and hoping for a valued outcome. Technologies defined in this patent application permit experimentation with simple projectiles hosting combinations.

Projectiles delivering unnatural Amino Acids (UAA) inside cells for protein production is included. Due to the physical sizes of the projectiles, new designs and processes are required to achieve a product.

The other extremes are applications wherein the depth of penetration is extremely small, maybe as small as a fraction of a millimeter, all cells are targets, and all projectiles are essentially identical. It is also possible that the trajectory of these projectiles can be uncontrolled (spherical or nearly spherical coverage). Pharmaceutical facilities using UAA to target thin film biological matter, an assembly line process, could be representative of this application.

Between cancer and thin film biological pharmaceutical applications are many other applications with unique requirements of penetration depth, target accuracy, and target quantities, and restrictions of various natures. Some restrictions likely to be encountered are; solid bone such as skull, nervous systems tissues requiring caution, any number of soft tissue sensitivity including major blood vessels, known risk factors for blood loss, most known medical issues found in patients with complications due to other illnesses beyond the one being treated by penetrating microscopic and/or nanoscopic projectiles. Like all medical treatments the attending physician(s) judgment will be part of the treatment's selection process, this also will become more complex as more, and different, projectile content is made available.

We claim:

1. A process for injecting a nanoparticle into a target cell of a plant, animal or hybrid plant-animal comprising the step of propelling the nanoparticle with sufficient momentum to penetrate the target cell.

2. A process according to claim 1 wherein the nanoparticle penetrates the cell without using the organism's circulatory system.

3. A process according to claim 1 wherein the target cell comprises cell membranes and the nanoparticle penetrates the cell membranes.

4. A process according to claim 1 wherein a central force accelerator propels the nanoparticle.

5. A process according to claim 1 wherein the nanoparticle is propelled with sufficient momentum to penetrate one or more cells before penetrating the target cell.

6. A process according to claim 4 wherein cells between the accelerator and the target cell will experience puncture damage as the nanoparticle transits en route to the target cell.

7. A process according to claim 1 further comprising the step of influencing the behavior of the cell.

8. A process according to claim 1 further comprising the step of influencing the future behavior of the cell.

9. A process for injecting nanoparticles into target cells of a plant, animal or hybrid plant-animal comprising the step of propelling the nanoparticles with sufficient momentum to penetrate the target cells.

10. A process according to claim 9 wherein nanoparticles previously not deemed medically useful or medically allowed when administered intravenously, now become medically useful.

11. A system for effecting cellular functionality in plants, and hybrid plant-animals, wherein nanoparticles are directly injected into the target cells, projectiles are propelled from a central force accelerator, said accelerator provides sufficient momentum to the projectiles to penetrate one or more cells resulting in deposition at a target cell.

12. A system according to claim 11 wherein the cellular functions include: altering the viability of the cell, altering the genetic composition of the cell to include providing genetic materials to correct known genetic errors, altering the genetic composition of the cell to add functions to the cell.

13. A system according to claim 12 wherein the cell is killed by an approved cancer drug, a chemical compound known to generate a malfunction in the cell, a segment of genetic material known to generate a malfunction in the cell.

14. A system according to claim 13 wherein the chemical compound generates an endothermic reaction within the cell, the chemical compound generates an exothermic reaction within the cell, the chemical compound generates a pressure producing reaction to burst the cell, the chemical compound stops an organelle from functioning.

15. A system according to claim 12 wherein the cell genetic material in the nucleus is augmented with segments of DNA, causing permanent genetic change.

16. A system according to claim 12 wherein the cell genetic material in the cytoplasm is augmented with segments of mRNA, tRNA, causing temporary genetic change.

17. A system according to claim 11 wherein nanoparticles are biological, including Ferritins, hosing compounds including: genetic material, chemotherapy drugs, chemical compounds.

18. A system according to claim 11 wherein nanoparticles re industrially produced, including single-walled carbon nanotubes, multiwalled carbon nanotubes, boron nitride nanotubes, metal plated nanotubes, nanotubes with end-effectors.

19. A system according to claim 11 wherein the central force accelerator is a rotational motion, a gyrational motion, further these accelerators have smoothness of acceleration surfaces that is near atomic reduce asperities to be smaller than one nanometer.

20. A system according to claim 19 wherein the smoothness of accelerator's surface in contact with nanoparticles being accelerated are composed of carbon nanotubes, boron nitride nanotubes, Graphene, and other materials having near two-dimensional surface characterized represented by graphene.

21. A system according to claim 11 wherein the nanoparticles have shapes to penetrate subcellular organelles; spherical, tubular, tubular with end-effector having piercing points smaller than one nanometer.
